(19) United States
(12) Patent Application Publication
(10) Pub. No.: US 2021/0038501 A1
(43) Pub. Date: Feb. 11, 2021

(54) MATERIALS AND METHODS FOR GENE DELIVERY IN THE HEART

(71) Applicant: Northwestern University, Evanston, IL (US)

(72) Inventors: Rishi Arora, Chicago, IL (US); David A. Johnson, Evanston, IL (US)

(21) Appl. No.: 16/773,540

(22) Filed: Jan. 27, 2020

Related U.S. Application Data
(60) Provisional application No. 62/961,514, filed on Jan. 15, 2020.
(60) Provisional application No. 62/947,737, filed on Dec. 13, 2019.
(60) Provisional application No. 62/942,516, filed on Dec. 2, 2019.
(60) Provisional application No. 62/884,012, filed on Aug. 7, 2019.

(51) Int. Cl. A61K 9/00; A61K 31/711

(52) U.S. Cl. CPC A61K 9/0009; A61K 31/711

(57) ABSTRACT

Provided herein are materials, methods, and devices for the targeted delivery of agents. In particular, provided herein are materials, methods, and devices for the targeted delivery of agents to the atria or ventricles of the heart.

### PRIORITY DATA

This application claims priority to U.S. Provisional Patent Application No. 62/884,012, filed Aug. 7, 2019, U.S. Provisional Patent Application No. 62/942,516, filed Dec. 2, 2019, U.S. Provisional Patent Application No. 62/947,737, filed Dec. 13, 2019, and U.S. Provisional Patent Application No. 62/961,514, filed Jan. 15, 2020, the entire contents of each of which are incorporated herein by reference.

### FIELD

The present disclosure relates to targeted gene delivery. In particular, provided herein are materials, methods, and devices for targeted gene delivery in the heart.

### BACKGROUND

Atrial fibrillation (AF) is the most common heart rhythm disorder, affecting more than 4 million Americans. It is also a major cause of stroke. The annual cost of AF in the US is >$6 billion. The diagnosis and management of AF have therefore become important and challenging aspects of cardiovascular medicine.

Gene therapy may be a viable option for treatment of disorders such as AF. However, systemic gene delivery often results in sub-therapeutic concentrations of a gene in the organ of interest. In addition, systemic delivery carries the risk of unwanted gene expression in organs that are remote from the region of interest, with the potential for significant side effects. However, localized gene therapy directly to the heart is often unsuccessful due to lack of adequate gene transfer into cardiomyocytes. Accordingly, novel methods for safe and effective gene-based therapies for the treatment of cardiac disorders such as AF are needed.

### SUMMARY

Provided herein are materials, methods, and devices for the targeted delivery of agents. In some aspects, provided herein are methods for delivering an agent to a subject. The methods include delivering the agent to a segment of the coronary vasculature of the subject and electroporating a target coronary tissue of the subject. In some aspects, provided herein are methods of treating a cardiac disorder in a subject. The methods for treating a cardiac disorder in a subject include delivering an agent to a segment of the coronary vasculature of the subject and electroporating a target coronary tissue of the subject. The cardiac disorder may be a heart arrhythmia, congestive heart failure, or coronary artery disease.

In some aspects, the segment of the coronary vasculature is different from the target coronary tissue. For example, the segment of the coronary vasculature may be the aortic root, the coronary artery, or the coronary sinus. The target coronary tissue may be the left atrium, the right atrium, the left ventricle, or the right ventricle. In some aspects, electroporation is performed prior to delivery of the agent, concurrently with delivery of the agent, and/or following delivery of the agent to the segment of the coronary vasculature of the subject. Electroporation may be performed by epicardial or endocardial electroporation.

In some aspects, the agent comprises a therapeutic agent for the treatment of a cardiac disorder in the subject. For example, the agent may include a nucleotide, an oligonucleotide, a protein, a peptide, a small molecule, or a macromolecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows Coomassie blue staining in the right atrium following injection into the aortic root and simultaneous electroporation from a multipolar basket catheter (64 poles) placed in the right atrium. No evidence of any significant staining in the left atrium is shown.

FIG. 2 shows an angiograph following retrograde coronary sinus injection of Coomassie blue dye. The electroporation basket catheter is seen in the right atrium.

FIG. 3 Coomassie blue staining in the right atrium following injection into the coronary sinus and simultaneous electroporation from a multipolar basket catheter (64 poles) placed in the right atrium. The left atrium (which was not electroporated) does not show significant staining.

FIG. 4 shows the left and right ventricles following injection into the coronary sinus and simultaneous electroporation from a multipolar basket catheter (64 poles) placed in the right atrium. The left and right ventricles (which were not electroporated) do not show significant staining.

FIG. 5A-5C show GFP expression in atrial tissue following injection of GFP expressing plasmid into the right atrium and endocardial electroporation of the right atrium. As shown in FIG. 5A-B, GFP expression was noted in the electroporated atrium. Furthermore, the GFP expression was found to be transmural (i.e. epicardial to endocardial expression). As shown in FIG. 5C, GFP expression was not noted in the non-electroporated atrium (e.g. the left atrium).

FIG. 6 shows the FirMap catheter placed in the right atrium (arrow), and coronary sinus injection of contrast dye.

FIG. 7A-7B shows Coomassie blue staining following injection and electroporation. Coomassie blue staining was only seen in the electroporated atrium. FIG. 7A shows Coomassie blue in right but not left atrium. FIG. 7B shows Coomassie blue in left but not right atrium.

FIG. 8 shows GFP expression following injection and electroporation. As shown in the figure, GFP expression is localized to the region of electroporation i.e. RAFW H and RAFW M. There is no GFP expression in the RAFW L, RAA and PLA. (RAFW—right atrial free well. H—high; M—mid; L—low. RAA—right atrial appendage. PLA—posterior left atrium. Endo—endocardium; Mid—mid myocardium; Epi—epicardium)

FIG. 9 is a western blot showing GFP expression. As shown, GFP expression is localized to the region of electroporation. (RAFW—right atrial free well. H—high; M—mid; L—low. RAA—right atrial appendage. PRA—posterior right atrium.)

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a nanocarrier” is a reference to one or more nanocarriers and equivalents thereof known to those skilled in the art, and so forth.

As used herein, the term “about,” when referring to a value is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “coronary vasculature” refers to the blood vessels responsible for coronary circulation, supplying blood to the heart muscle (myocardium). The term “blood vessels” includes both arteries and veins. “Coronary arteries” supply oxygenated blood to the heart muscle, and “cardiac veins” drain away the blood once it has been deoxygenated.

The term “gene therapy” is given its ordinary meaning in the art. Briefly, “gene therapy” refers to the transfer of genetic material (e.g., a DNA or RNA) of interest into a host cell and/or tissue. The genetic material of interest typically encodes a product whose in vivo production is desired. The genetic material of interest can also include various control elements, such as transcriptional promoters. It is noted that the end result of gene therapy does not have to always include a cure, but instead, also includes reducing the severity of one or more symptoms of a disease.

As used herein, the term “subject” refers to any animal including, but not limited to, insects, humans, non-human primates, vertebrates, bovines, equines, felines, canines, pigs, rodents, and the like. The terms “subject” and “patient” may be used interchangeably. A subject may be of any stage of life (e.g. embryo, fetus, infant, neonatal, child, adult, etc.). A subject may be male or female.

As used herein, the terms “treat,” “treatment,” and “treating” refer to reducing the amount or severity of a particular condition, disease state (e.g., cardiovascular disorder), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

### DETAILED DESCRIPTION

In some embodiments, provided herein are devices and methods for the targeted delivery of agents (e.g. nucleic acids, gene therapy agents, etc.) to a subject. The methods comprise delivering the agent to a segment of the coronary vasculature of the subject, and electroporating a target coronary tissue of the subject. Electroporation of the target coronary tissue of the subject results in targeted delivery of the agent to (or within) the target coronary tissue.

In some embodiments, the segment of the coronary vasculature is different from the target coronary tissue. For example, the segment of the coronary vasculature may be a suitable artery or vein for injecting the agent and the target coronary tissue may be a different tissue wherein the localized distribution of the agent is intended to occur.

The segment of the coronary vasculature and the target coronary tissue may be selected for targeted delivery of the agent to the cardiovascular system. Such methods would be useful for treatment of cardiac disorders, including Atrial Fibrillation. Atrial fibrillation (AF) is the most common heart rhythm disorder. It affects >3 million Americans and is a major cause of stroke. Since AF is primarily an age-related disease, it is fast becoming an epidemic in an aging population. Unfortunately, current therapies for AF—both pharmacological and ablation-based—are sub-optimal in patients with persistent AF. This is thought to be in part because current treatments do not target the fundamental, molecular mechanisms that cause AF.

In some embodiments, provided herein is an approach to AF treatment that targets one or more molecular mechanisms underlying development of the AF disease state. In some embodiments, the devices and methods herein target the underlying mechanisms of AF via delivery of an agent. In certain embodiments, devices and methods herein target the underlying mechanisms of AF via delivery of a nucleic acid. In particular embodiments, devices and methods herein target the underlying mechanisms of AF via delivery of a nucleic acid gene therapy agent.

In some embodiments, provided herein are devices and methods for the targeted delivery of agents to the heart. In some embodiments, the devices and methods disclosed herein may be used for the treatment of a cardiac disorder. For example, provided herein are devices and methods for the targeted delivery of an agent to the atrium, such as for the treatment of atrial fibrillation. As another example, provided herein are devices and methods for the targeted delivery of an agent to the ventricle, such as for the treatment of a ventricular arrhythmic disorder. In some embodiments, the devices herein encompass injection and electroporation technologies (e.g., array-based electroporation) for a precise and targeted delivery of the agent into the desired tissue (e.g., atrium, ventricle). In some embodiments, the devices are capable of delivering one or multiple agents (e.g., nucleic acids (e.g., trans-genes)) into the desired tissue in a precise amount so that potential toxicities are avoided.

In some embodiments, devices and methods herein utilize electroporation or sonoporation to achieve gene delivery in the intended tissue (e.g., the atrium, the ventricle, etc.). Many embodiments here are described in connection with electroporation; however, any such embodiments may also find use with sonoporation or other techniques for achieving acceptance of a therapeutic (e.g., nucleic acid therapeutic) into cells or tissues. Both viral and non-viral vectors may be used for cardiac gene delivery. Viruses can be advantageous vectors due to long term gene expression. However, viral vectors have potential for off-target effects. Accordingly, non-viral delivery approaches (e.g., plasmids, cosmids, etc.) may also be used in accordance with the methods described herein.

In some embodiments, use of electroporation or sonoporation to deliver the agent to the intended tissue nearly eliminates the possibility of off-target effects, as gene expression is localized to the site of electroporation/sonoporation. Plasmid DNA is rapidly degraded in blood and has no mechanism to transfect other cells after IV injection. In some embodiments, these advantages also obviate the need for organ- or tissue-specific promoters (e.g., cardiac specific promoters). Furthermore, a physical method such as electroporation may significantly enhance even viral gene transfection (e.g., in the atrium). In some embodiments, devices and methods herein utilize electroporation-facilitated non-viral agent (e.g., nucleic acid (e.g., trans-gene)) delivery.

In some embodiments, provided herein are methods of treating a cardiac disorder in a subject, comprising delivering an agent to a segment of the coronary vasculature of the subject, and electroporating a target coronary tissue of the subject. In some embodiments, the agent is delivered (e.g., passively or actively) via the vasculature to the target coronary tissue. In some embodiments, the present invention provides treatment or prevention of a cardiac disorder or condition selected from the list of aortic dissection, cardiac arrhythmia (e.g. atrial cardiac arrhythmia (e.g. premature atrial contractions, wandering atrial pacemaker, multifocal atrial tachycardia, atrial flutter, atrial fibrillation, etc.), junctional arrhythmias (e.g. supraventricular tachycardia, AV nodal reentrant tachycardia, paroxysmal supra-ventricular tachycardia, junctional rhythm, junctional tachycardia, premature junctional complex, etc.), atrio-ventricular arrhythmias, ventricular arrhythmias (e.g. premature ventricular contractions, accelerated idioventricular rhythm, monomorphic ventricular tachycardia, polymorphic ventricular tachycardia, ventricular fibrillation, etc.), congenital heart disease, myocardial infarction, dilated cardiomyopathy, hypertrophic cardiomyopathy, aortic regurgitation, aortic stenosis, mitral regurgitation, mitral stenosis, Ellis-van Creveld syndrome, familial hypertrophic cardiomyopathy, Holt-Orams Syndrome, Marfan Syndrome, Ward-Romano Syndrome, and/or similar diseases and conditions. In some embodiments, the cardiac disorder may be any one of more of a heart arrhythmia, congestive heart failure, and coronary artery disease. For example, the cardiac disorder may be a heart arrhythmia, such as an atrial arrhythmia or a ventricular arrhythmia. The arrythmia may be a tachycardia or a bradycardia. Exemplary arrhythmias include, for example, atrial fibrillation, atrial flutter, supraventricular tachycardia, Wolf-Parkinson-White syndrome, ventricular tachycardia, ventricular fibrillation, long QT syndrome, sick sinus syndrome, conduction block, and the like.

The agent may be administered by any suitable route. The route of administration will depend upon the intended location of delivery of the agent within the subject. For example, the agent may be administered by catheter-based delivery methods, needle-based delivery methods, non-needle-based delivery methods, laparoscopically, surgically (e.g. by open-heart surgery), systemically (e.g. enteral or parenteral administration), topically, or by an injection apparatus. Exemplary apparatuses are described in U.S. Patent Application Publication No. 20110245756 and U.S. Patent Application Publication No. 2011013728, each of which are incorporated herein by reference in their entirety. In some embodiments, the agent is administered to the segment of the coronary vasculature using catheter-based injection methods.

In particular embodiments, the mode of administration is selected to avoid open-heart surgery. For example, the agent may be delivered using a catheter inserted through a site in the body separate from the heart. The catheter may be inserted into any suitable body part and guided to the segment of the coronary vasculature prior to administration of the agent to the segment of the coronary vasculature of the subject. For example, the catheter may be inserted into a vein or artery in a body part such as the leg, groin, armpit, and the like to allow for delivery of the agent to a desired location within the heart without the need for open-heart surgery.

The agent may be administered endocardially or epicardially. For example, the agent may be injected into the atrium or the ventricles by endocardial or epicardial injection. In some embodiments, the agent is administered by intracoronary injection. Intracoronary injection encompasses injecting the agent to any suitable area of the heart (e.g. artery, vein, sinus) without the need for direct application or injection to the atria or ventricles. Accordingly, in some embodiments, the segment of the coronary vasculature may be the coronary veins that go into the coronary sinus (e.g., the great cardiac vein, the middle cardiac vein, the small cardiac vein, the posterior vein of the left ventricle, the vein of Marshall, etc.), coronary veins that go directly to the right atrium (e.g., the anterior cardiac veins, the smallest cardiac veins (Thebesian veins), etc.), aorta, the aortic root, the coronary artery (e.g. the right coronary artery, the left main coronary artery, the circumflex artery, the left anterior descending artery), the left marginal artery, the right marginal artery, the posterior descending artery, the coronary sinus, the vena cava (e.g. superior vena cava, inferior vena cava), a pulmonary vein (e.g. right pulmonary veins, left pulmonary veins), a pulmonary artery (left pulmonary arteries, right pulmonary arteries), the brachiocephalic artery, the carotid artery, the subclavian artery, the pericardial space, or combinations thereof. In some embodiments, the segment of the coronary vasculature is selected to allow for non-invasive delivery of the agent to the subject (e.g. injection without the need for open-heart surgery, such as catheter-based techniques). In some embodiments, the segment of the coronary vasculature is selected from the aortic root, the coronary artery, the coronary sinus, and combinations thereof.

In some embodiments, one or more areas may be occluded during administration of the agent to the segment of the coronary vasculature. For example, one or more areas in the heart or coronary vasculature may be occluded to prevent flow of the agent to unintended tissues. Exemplary occlusion methods include, for example, balloon occlusion. As with the administration of the agent, the occlusion procedure may be performed without the need for open-heart surgery. Any suitable area may be occluded as needed to prevent the flow of the agent to unintended tissues, including one or more arteries or veins within the heart. As one example, the agent may be injected into the coronary artery and the coronary sinus may be occluded (such as by balloon occlusion). As another example, the agent may be injected in the aortic root and the proximal aorta may be occluded. In some embodiments, occlusion prevents the agent from travelling to unintended portions of the coronary vasculature and/or contacting unintended tissues. In some embodiments, occlusion allows the agent to move passively by diffusion, rather than being moved by the blood flow within the coronary vasculature.

In some embodiments, the target coronary tissue (e.g. the tissue where localized distribution of the agent is intended to occur) is one or more of the left atrium, the right atrium, the left ventricle, and the right ventricle. For example, for methods of treating an atrial arrythmia, the target coronary tissue may be the left and/or right atrium. As another example, for methods of treating a ventricular arrythmia, the target coronary tissue may be the left and/or right ventricle.

The methods described herein further comprise electroporating or sonoporating a target coronary tissue. The electroporation or sonoporation may be performed prior to delivery of the agent, concurrently with delivery of the agent, and/or following delivery of the agent to the segment of the coronary vasculature of the subject. For example, electroporation or sonoporation may be performed less than 1 hour prior to delivery of the agent. For example, electroporation or sonoporation may be performed less than 1 hour, less than 55 minutes, less than 50 minutes, less than 45 minutes, less than 40 minutes, less than 35 minutes, less than 30 minutes, less than 25 minutes, less than 20 minutes, less than 15 minutes, less than 10 minutes, less than 5 minutes, less than 4 minutes, less than 3 minutes, less than 2 minutes, less than 1 minute, less than 45 seconds, less than 30 seconds, less than 15 seconds, less than 10 seconds, less than 5 seconds, or less than 1 second prior to delivery of the agent. Alternatively or in combination, electroporation or sonoporation may be performed concurrently with delivery of the agent. Alternatively or incombination, electroporation or sonoporation may be performed following delivery of the agent. For example, electroporation or sonoporation may be performed less than 1 second, less than 5 seconds, less than 10 seconds, less than 15 seconds, less than 30 seconds, less than 45 seconds, less than 1 minute, less than 2 minutes, less than 3 minutes, less than 5 minutes, less than 5 minutes, less than 10 minutes, less than 15 minutes, less than 20 minutes, less than 25 minutes, less than 30 minutes, less than 35 minutes, less than 40 minutes, less than 45 minutes, less than 50 minutes, less than 55 minutes, or less than 1 hour following delivery of the agent.

Electroporation or sonoporation may be performed any suitable number of times for any suitable duration to achieve the desired effect. For example, electroporation or sonoporation may be performed once or more than once. Electroporation may be performed endocardially or epicardially. For example, electroporation may be performed by epicardial electroporation. Alternatively or in combination, electroporation may be performed by endocardial electroporation. Any suitable device for electroporation or sonoporation may be used. For example, electroporation may be performed with closely spaced, bipolar electrodes. Alternatively, electroporation can be performed with either a bipolar or a multipolar catheter. An example of a multipolar catheter that can help facilitate endocardial electroporation is a commercially available Basket catheter. Such a catheter typically covers almost the entire surface area of a single atrium. Electroporation from such a catheter could therefore be performed in a way that an entire atrium can be subjected to electroporation during the process of intracoronary gene injection. This would allow selective gene transfer to occur in the entire atrial territory where electroporation is being performed. Alternatively, electroporation of the entire atrium may be performed by positioning a catheter (such as a multipolar catheter) in a first position of the atrium and electroporating the tissue, moving the catheter to a second position and electroporating the tissue, moving the catheter to a third position and electroporating the tissue, etc. until the agent has been delivered to the entire atrium. Using this method, any catheter, including a small catheter, can be sufficient to deliver the agent to the entire atrium.

In some embodiments, separate devices may be used for delivery of the agent of the segment of the coronary vasculature and electroporation or sonoporation of the target coronary tissue. In other embodiments, the same device may be used for delivery and electroporation or sonoporation. Suitable devices are described in U.S. Patent Application Publication No. 20110245756 and U.S. Patent Application Publication No. 2011013728, each of which are incorporated herein by reference in their entirety.

Electroporation, or electropermeabilization, refers to a significant increase in the electrical conductivity and permeability of the cell plasma membrane caused by an externally applied electrical field. Any suitable level of electric current can be delivered to the target coronary tissue within a subject. In some embodiments, the level of electric current applied to the tissue is selected based on the subject (e.g., species, size, age, etc.), treatment site (e.g., epicardium, endocardium, etc.), and other considerations known to those of skill in the art. In some embodiments, electric current is delivered continuously. The electric current may be delivered continuously for any suitable period of time. For example, the electric current may be delivered for 1 microsecond to 1 hour. For example, the electric current may be administered for 1 microsecond, 10 microseconds, 50 microseconds, 100 microseconds, 150 microseconds, 200 microseconds, 250 microseconds, 300 microseconds, 350 microseconds, 400 microseconds, 450 microseconds, 500 microseconds, 550 microseconds, 600 microseconds, 650 microseconds, 700 microseconds, 750 microseconds, 800 microseconds, 850 microseconds, 900 microseconds, 950 microseconds, 1000 microseconds, 10 milliseconds, 20 milliseconds, 30 milliseconds, 40 milliseconds, 50 milliseconds, 60 milliseconds, 70 milliseconds, 80 milliseconds, 90 milliseconds, 100 milliseconds, 150 milliseconds, 200 milliseconds, 250 milliseconds, 300 milliseconds, 350 milliseconds, 400 milliseconds, 450 milliseconds, 500 milliseconds, 550 milliseconds, 600 milliseconds, 650 milliseconds, 700 milliseconds, 750 milliseconds, 800 milliseconds, 850 milliseconds, 900 milliseconds, 950 milliseconds, 1 second, 2 seconds, 5 seconds, 10 seconds, 30 seconds, 1 minute, 2 minutes, 5 minutes, 10 minutes, 30 minutes, 1 hour, or more).

In some embodiments, electric current is pulsed. The length of the pulse, the current applied, and the duration of pulsing may be selected based on appropriate criteria determined by a skilled artisan or clinician. In some embodiments, pulses are 1 microsecond second to 10 seconds in length. For example, electric current may be delivered in pulses of 1 microsecond, 10 microseconds, 50 microseconds, 100 microseconds, 150 microseconds, 200 microseconds, 250 microseconds, 300 microseconds, 350 microseconds, 400 microseconds, 450 microseconds, 500 microseconds, 550 microseconds, 600 microseconds, 650 microseconds, 700 microseconds, 750 microseconds, 800 microseconds, 850 microseconds, 900 microseconds, 950 microseconds, 1000 microseconds, 10 milliseconds, 20 milliseconds, 30 milliseconds, 40 milliseconds, 50 milliseconds, 60 milliseconds, 70 milliseconds, 80 milliseconds, 90 milliseconds, 100 milliseconds, 150 milliseconds, 200 milliseconds, 250 milliseconds, 300 milliseconds, 350 milliseconds, 400 milliseconds, 450 milliseconds, 500 milliseconds, 550 milliseconds, 600 milliseconds, 650 milliseconds, 700 milliseconds, 750 milliseconds, 800 milliseconds, 850 milliseconds, 900 milliseconds, 950 milliseconds, 1 second, 2 seconds, 5 seconds, or 10 seconds. Pulses may be spaced by any suitable amount of time (e.g. microsecond to 10 seconds). For example, pulses may be 1 microsecond, 10 microseconds, 50 microseconds, 100 microseconds, 150 microseconds, 200 microseconds, 250 microseconds, 300 microseconds, 350 microseconds, 400 microseconds, 450 microseconds, 500 microseconds, 550 microseconds, 600 microseconds, 650 microseconds, 700 microseconds, 750 microseconds, 800 microseconds, 850 microseconds, 900 microseconds, 950 microseconds, 1000 microseconds, 10 milliseconds, 20 milliseconds, 30 milliseconds, 40 milliseconds, 50 milliseconds, 60 milliseconds, 70 milliseconds, 80 milliseconds, 90 milliseconds, 100 milliseconds, 150 milliseconds, 200 milliseconds, 250 milliseconds, 300 milliseconds, 350 milliseconds, 400 milliseconds, 450 milliseconds, 500 milliseconds, 550 milliseconds, 600 milliseconds, 650 milliseconds, 700 milliseconds, 750 milliseconds, 800 milliseconds, 850 milliseconds, 900 milliseconds, 950 milliseconds, 1 second, 2 seconds, 5 seconds, or 10 seconds apart. Any suitable number of pulses may be delivered to a tissue within a desired time frame. In some embodiments, pulses may be delivered for a total of 1 s to 1 hour, counting the duration of each pulse and each space between pulses. For example, the pulses may be delivered for a total of 1 second, 2 seconds, 5 seconds, 10 seconds, 30 seconds, 1 minute, 2 minutes, 5 minutes, 10 minutes, 30 minutes, 45 minutes, or 1 hour).

In some embodiments, the level of electric current applied is between 1 Volt and 1000 Volts. For example, the electric current may be 1 Volt, 2 Volts, 3 Volts, 4 Volts, 5 Volts, 6 Volts, 7 Volts, 8 Volts, 9 Volts, 10 Volts, 15 Volts, 20 Volts, 25 Volts, 30 Volts, 35 Volts, 40 Volts, 45 Volts, 50 Volts, 55 Volts, 60 Volts, 65 Volts, 70 Volts, 75 Volts, 80 Volts, 85 Volts, 90 Volts, 95 Volts, 100 Volts, 150 Volts, 200 Volts, 250 Volts, 300 Volts, 350 Volts, 400 Volts, 450 Volts, 500 Volts, 550 Volts, 600 Volts, 650 Volts, 700 Volts, 750 Volts, 800 Volts, 850 Volts, 900 Volts, 950 Volts, or 1000 Volts.

Sonoporation, or cellular sonication, is the use of sound (e.g., ultrasonic frequencies) for modifying the permeability of the cell plasma membrane. In some embodiments, a device of the present invention directs sonic energy (e.g., ultrasound frequencies) to a treatment site to aid in therapeutic (e.g., nucleic acid) uptake. In some embodiments, any suitable level of ultrasound can be delivered through a device of the present invention and applied to a site within a subject. In some embodiments, the level and/or frequency of ultrasound applied to a site (e.g. treatment site, delivery site, etc.) is selected based on the subject (e.g., species, size, age, etc.), treatment site (e.g., epicardium, endocardium, etc.), and other considerations known to those of skill in the art.

In some embodiments, ultrasound is delivered continuously for a period of time (e.g., 1 second, 2 seconds, 5 seconds, 10 seconds, 30 seconds, 1 minute, 2 minutes, 5 minutes, 10 minutes, 30 minutes, 1 hour, or more). In some embodiments, ultrasound is pulsed. In some embodiments, the length of pulse, level and/or frequency of ultrasound applied, and duration of pulsing are selected based on appropriate criteria determined by a skilled artisan or clinician. In some embodiments, the frequency of ultrasound applied by a device of the present invention is between 20 kHz and 200 MHz (e.g., 20 kHz, 50 kHz, 100 kHz, 200 kHz, 500 kHz, 1 MHz, 2 MHz, 5 MHz, 10 MHz, 20 MHz, 50 MHz, 100 MHz, 200 MHz). In some embodiments, the level of ultrasound applied by a device of the present invention has a mechanical index (MI) between 0.01 and 5 (e.g., 0.01, 0.02, 0.05, 0.1, 0.2, 0.5, 1.0, 2.0, 5.0). In some embodiments, pulses are 0.1 seconds to 10 seconds in length (e.g., 0.1 s, 0.2 s, 0.5 s, 1 s, 2 s, 5 s, 10 s), and delivered for 1 s to 1 hour (e.g., 1 second, 2 seconds, 5 seconds, 30 seconds, 1 minute, 2 minutes, 5 minutes, 10 minutes, 30 minutes, 1 hour).

The electroporation or sonoporation device may be provided at the target coronary tissue without the need for open-heart surgery. For example, the electroporation at the target coronary tissue site may be performed using a catheter-based electroporation device. For example, the electroporation device may be provided at the target coronary tissue using non-invasive, catheter-based methods. The catheter-based electroporation device may be inserted at in any suitable site in the body separate from the heart, such as a vein or an artery, and guided to the desired target coronary tissue. For example, the catheter-based electroporation device may be inserted through a vein or an artery in the leg, groin, arm, or any other suitable body area of the subject.

In some embodiments, the agent comprises a therapeutic agent (e.g. a biologic agent) for the treatment of a cardiac disorder in the subject. For example, the agent may be a nucleotide, an oligonucleotide, a protein, a peptide, a small molecule, or a macromolecule. In some embodiments, the agent is a nucleotide (e.g. DNA (e.g., plasmids, mini-genes, etc.), RNA (e.g., siRNA, shRNA, etc.). In some embodiments, the agent is a naked DNA plasmid. In other embodiments, the agent further comprises a carrier. For example, the carrier may be a vector. Any suitable vector may be used, including viral vectors (e.g. adenovirus, adeno-associated virus, alphavirus, herpesvirus, retrovirus, lentivirus, vaccinia virus, etc.) and non-viral vectors.

Fundamental mechanisms in the creation of the atrial fibrillation (AF) disease state and several trans-genes that selectively target these mechanisms in the atrium have been identified (Ref 1-3; incorporated by reference in their entireties). In some embodiments, the agent may be designed to target any one of more of these mechanisms that contribute to the underlying disease state (e.g. AF). In some embodiments, devices and methods herein target, either singly or in combination, two fundamental mechanisms that contribute to electrical remodeling in AF, oxidative stress and parasympathetic nervous system signaling. In some embodiments, nucleic acids (e.g., plasmids) expressing the following trans-genes are used: NOX2 shRNA (this transgene inhibits NOX2, a major enzymatic source of oxidative stress), and/or C-terminal Gαi+Gαo inhibitory peptides (these plasmids inhibit parasympathetic signaling in the atrium). In some embodiments, a subject is administered a biological product comprising a combination of NOX2 shRNA+Gαi expressing plasmid+Gαo expressing plasmid. NOX2 shRNA entirely prevents RAP-induced electrical remodeling (and AF). NOX2 shRNA also prevents atrial fibrosis in a HF model. Parasympathetic inhibition (with Gα_{i/o}-ct) also significantly attenuated RAP induced electrical remodeling and AF. NOX2 shRNA attenuated parasympathetic nerve sprouting in dogs undergoing RAP, indicating a significant interaction between oxidative injury and parasympathetic signaling in creation of electrical remodeling in AF. Furthermore, NOX2 shRNA reversed electrical remodeling in RAP dogs with established AF, especially when given in combination with Gα_{i/o}-ct.

In some embodiments, the agent may be a gene (e.g. DNA) with or without a vector. Suitable targets for gene therapy include any target that contributes to the cause of the cardiac disorder. For example, suitable targets for an atrial or ventricular arrhythmic disorder may include targets that contribute to shortened action potentials (e.g. ion channels, autonomic modulation) or delayed conduction (e.g. gap junctions, structural remodeling) that may contribute to the development of the disorder. For example, the agent may be an ion channel modulator. For example, the agent may be a gene that prolongates the atrial action potential (e.g. variants of KCNH2, variants of IKR subunits, etc.). As another example, the agent may target connexin biology, which is thought to be associated with impaired electrical conduction in the atrium (e.g. connexins 40 and 43). The agent may target local and systemic inflammation or the development of fibrosis. For example, the agent may target enzymes known to be involved with inflammation and/or apoptosis (e.g. calpain, caspase-3, SOD1, etc.). As another example, the agent may target factors known to be involved in fibrosis (e.g. TGF-beta) or other transcription factors known to impact the development of the cardiac disorder (e.g. PITX2).

Both sympathetic and parasympathetic activity in the heart is mediated by heterotrimeric G-protein (GαGα3Gα) coupled pathways initiated by G-protein coupled receptors (GPCRs). In some embodiments, the present invention provides a gene-based approach to selectively inhibit the G-protein signaling pathways. In some embodiments, the present invention is used in an epicardial approach to administer minigenes expressing G-protein inhibitory peptides to a tissue (e.g. atrium, ventricle) in order to selectively inhibit the C-terminus of Gαi and Gαs in this region. In some embodiments, the present invention provides electroporation and/or ultrasound energy to enhance the effectiveness of gene therapy (e.g., for naked DNA and/or viral vectors). In some embodiments, electroporation and/or ultrasound energy enhance intracellular gene transfer. In some embodiments, the present invention targets G-protein mediated autonomic signaling, and/or other key signal transduction pathways (e.g. the TGF-beta pathway in the creation of atrial fibrosis). In some embodiments, the present invention provides a targeted gene-based approach to attenuate TGF-beta signaling in the left atrium, in order to decrease the development of fibrosis in AF. In some embodiments, the present invention provides methods for blocking G protein coupled receptor mediated signaling for treating atrial fibrillation (see, U.S. application Ser. No. 12/430,595, herein incorporated by reference in its entirety).

The methods described herein may be used in combination with other suitable therapies for the treatment of cardiac disorder in the subject. For example, the methods described herein may be used in combination with other suitable therapies for the treatment of a heart arrythmia, such as anticoagulants (e.g. warfarin, non-vitamin K antagonist oral anticoagulants), beta blockers, calcium channel blockers, cardiac glycosides (e.g. digoxin) antiarrhythmic drug therapies, cardioversion, catheter ablation, or other surgical procedures to restore and maintain normal sinus rhythm.

The methods described herein may further include monitoring the patient's response to the agent. For example, the methods may further include monitoring the response to delivery of the agent after the agent is delivered to the segment of the coronary vasculature and/or after the target coronary tissue is electroporated. Suitable methods for measuring the patient's response may include measuring the cardiac response to the agent. For example, response may be measured by cardiac MRI imaging (may be used in combination with ECG gating), electrocardiography, photoplethysmography, echocardiogram, computed tomography, nuclear medicine scans, and the like. In some embodiments, delivery of the therapeutic agent and/or electroporation of the target coronary tissue may continue until a favorable response in the subject is measured. For example, delivery and/or electroporation may continue until the arrhythmia ceases in the subject (e.g. normal cardiovascular function is restored).

### EXAMPLES

The atrial well is very thin, and it can be very difficult to perform gene injection in a manner that is not only safe (i.e. does not cause perforation) but allows delivery of a sufficient volume/amount of gene in the atrial wall. Accordingly, described herein is a novel method to facilitate gene delivery in the heart—selectively in the atrium and/or the ventricle—without having to perform direct needle injection of gene into the desired location within the heart.

The following experiments were conducted in a canine model. For the following experiments, 20 ml-200 ml of Coomassie blue at a concentration of 0.2-0.4 mg/100 ml was injected. Electroporation was performed using 10-30 pulses of 75-200 Volts for 10 msec each. Pulses were spaced 1 second apart. Tissue was harvested 10 minutes to 2 hours following injection.

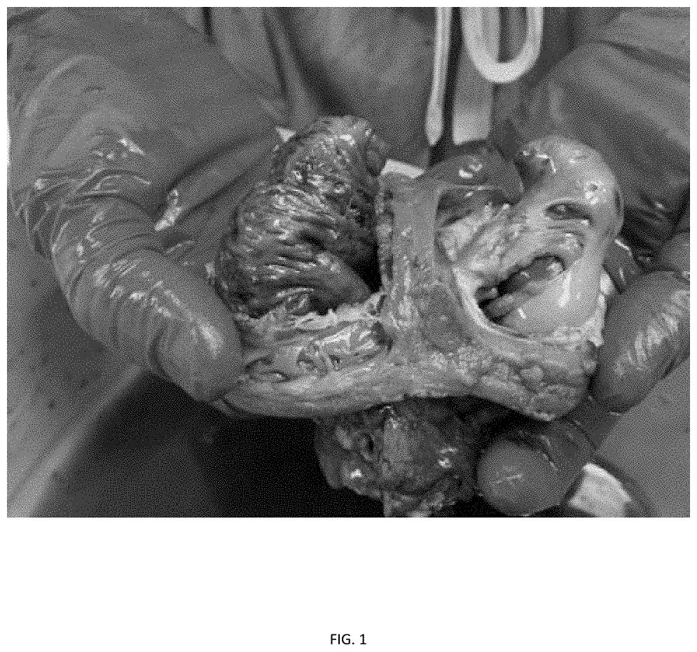

Experiment 1: In this experiment, electroporation from a multipolar ‘basket’ catheter (64 poles) placed in the right atrium and simultaneous injection of contrast containing color dye (Coomassie blue) in the aortic root (after clamping the proximal aorta with a Satinsky clamp) was performed. Results are shown in FIG. 1.

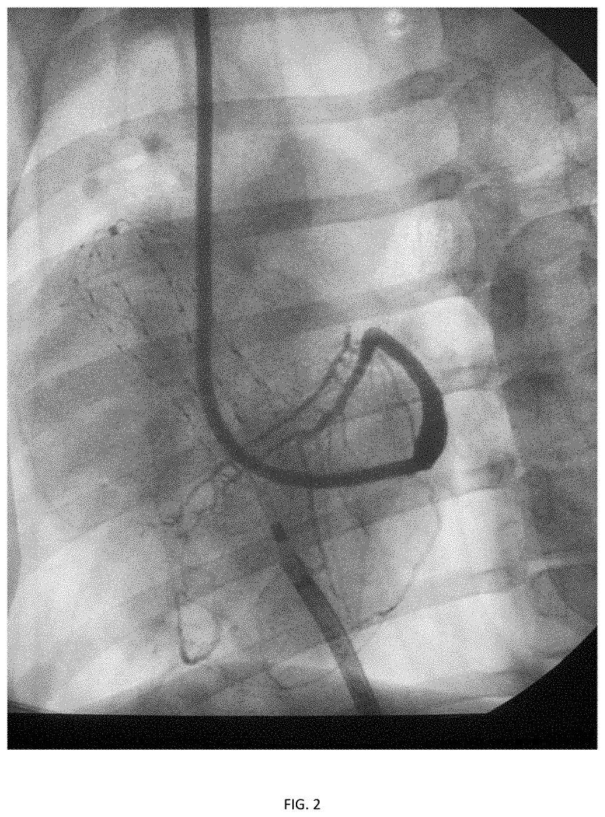

Experiment 2: In a follow-up experiment, electroporation from a multipolar ‘basket’ catheter (64 poles) placed in the right atrium and simultaneous injection of Coomassie blue in the retrograde coronary sinus. Results are shown in FIGS. 2-4. FIG. 2 shows an angiograph of retrograde coronary sinus injection of the Coomassie blue dye. The electroporation basket catheter is seen in the right atrium.

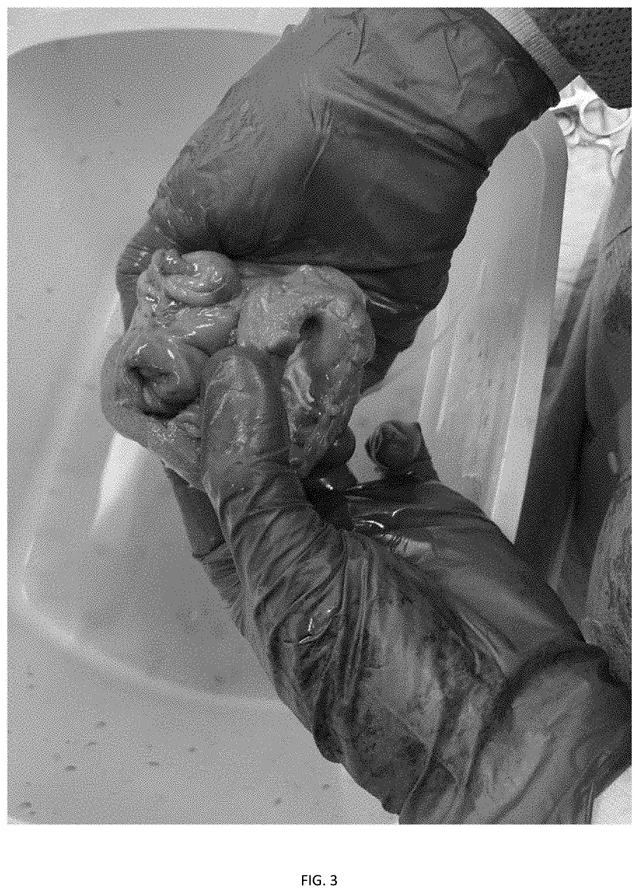

FIG. 3 shows that the right atrium was dyed with Coomassie blue after electroporation. Although the left atrium also received the Coomassie blue dye due to diffusion from coronary sinus, no significant Coomassie blue staining occurs because the left atrium was not electroporated.

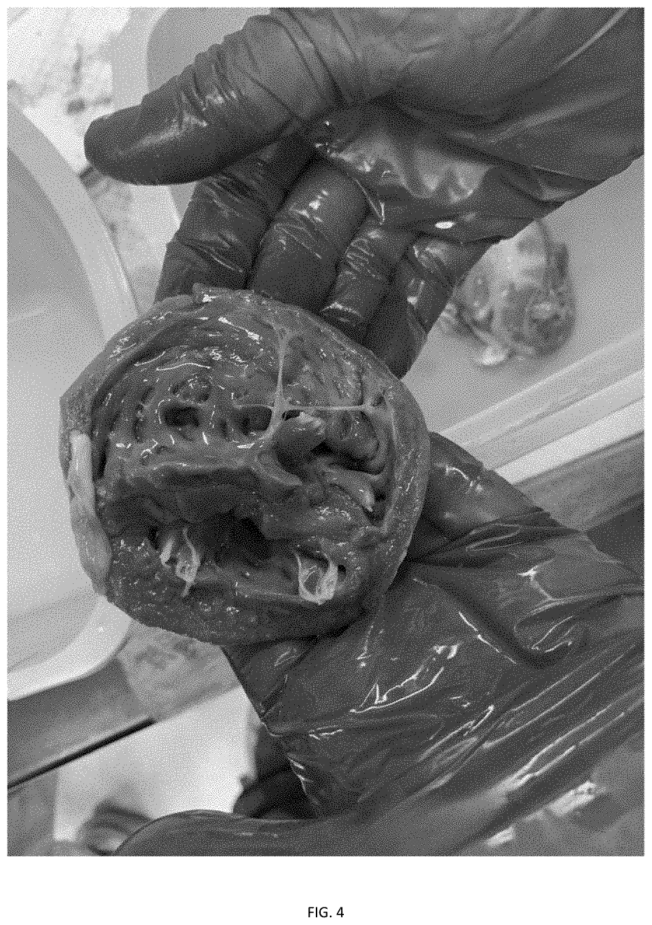

FIG. 4 shows that although the left and right ventricles also received Coomassie Blue dye via the retrograde coronary sinus injection, with no electroporation there is no significant Coomassie blue staining.

Further experiments were conducted to see whether the methods described herein are effective for gene delivery to the atrium. In one animal, the coronary sinus was cannulated via a jugular venous approach. Using a femoral venous approach, a FirMap catheter (64 electrodes; Abbott—St. Jude) was advanced into the high right atrium. Following balloon occlusion in the proximal coronary sinus, 1.5 mg of GFP expressing plasmid (under control of a CMV promoter) was diluted up to 20 ml and injected in the coronary sinus. While injection was being performed, electroporation was performed simultaneously in the high right atrium (encodardially) via the FirmMap catheter (Voltage—200V; Pulse duration—10 ms; Number of pulses—20; Interval between pulses—1 second). The gene injection and electroporation sequence was repeated three more times. After 3 days, the animal was sacrificed and the heart removed for further analysis.

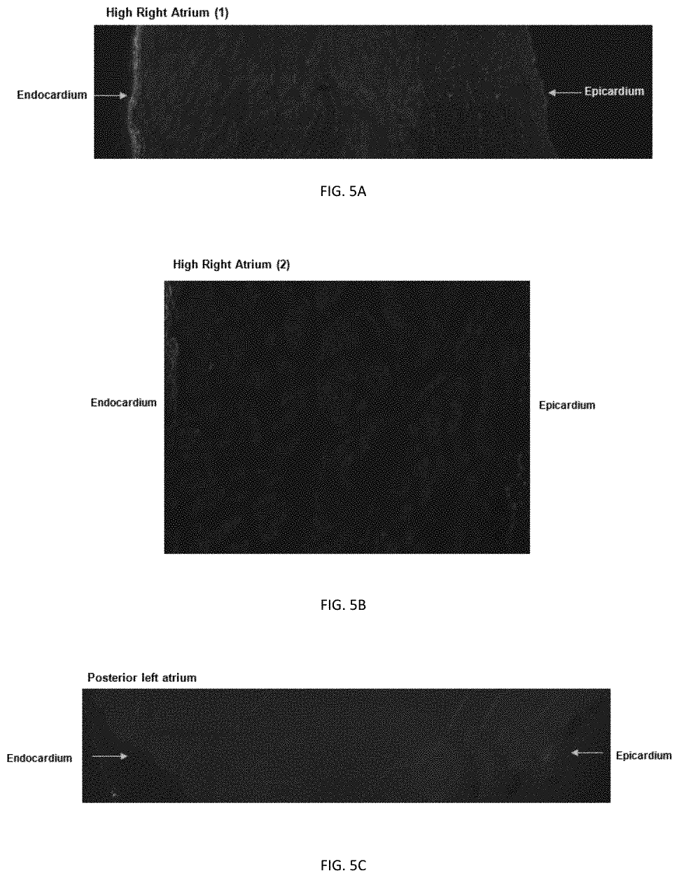

The electroporated high right atrium and non-electroporated posterior left atrium (control atrium) were examined for GFP expression using fluorescence microscopy. As shown in FIG. 5A-B, GFP expression was noted in the electroporated atrium. Furthermore, the GFP expression was found to be transmural (i.e. epicardial to endocardial expression). As shown in FIG. 5C, GFP expression was not noted in the non-electroporated atrium (e.g. the left atrium). These results demonstrate that it is possible to obtain robust gene expression in the atrium via this unique new ‘needleless’ method.

### Example 2

### Coronary Sinus Gene Delivery and Targeted, Simultaneous Atrial Electroporation—a New Trans-Venous Method to Obtain Atrial Gene Delivery

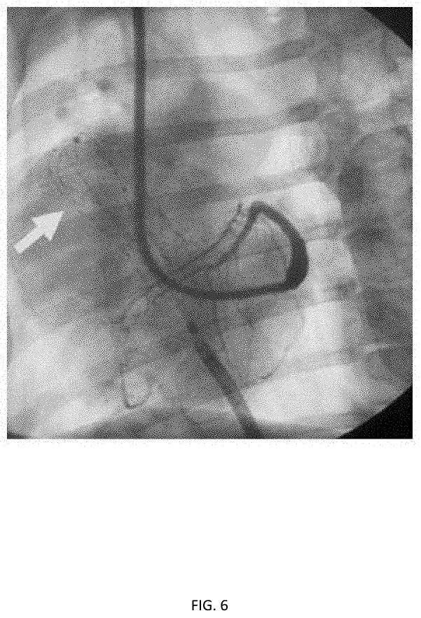

Coomassie blue injection: In two animals, the coronary sinus was cannulated via a jugular venous approach. In one animal, a FirMap catheter (64 electrodes; Abbott—St. Jude) was advanced into the high right atrium via a femoral venous approach. In the second animal, a FirMap catheter was advanced into the left atrium via a trans-septal puncture. In both animals, balloon occlusion was performed in the proximal coronary sinus, followed by coronary sinus injection of Coomassie blue dye (80 mg of dye diluted to 20 ml) mixed with contrast dye. While injection was being performed, electroporation was performed simultaneously in right or left atrium atrium via the FirMap catheter (Voltage—200V; Pulse duration—10 ms; Number of pulses—20; Interval between pulses—1 second). FIG. 6 shows the FirMap catheter in the high right atrium; the figure also shows coronary sinus injection of contrast dye. Each animal was sacrificed, and the atria examined for evidence of Coomassie blue uptake.

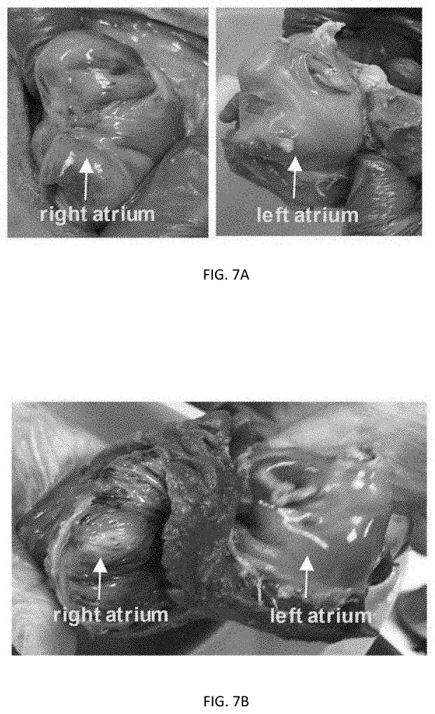

As shown in FIG. 7A, Coomassie blue was found only in the atrium in which electroporation was performed (i.e. the right atrium), with no dye present in the non-electroporated atrium (i.e. left atrium). In a second animal, trans-septal puncture was performed and electroporation was performed (during coronary sinus injection of Coomassie blue) in the posterior left atrium. As shown in FIG. 7B, Coomassie blue was found only in the left atrium (where electroporation was performed) with no dye in the right atrium.

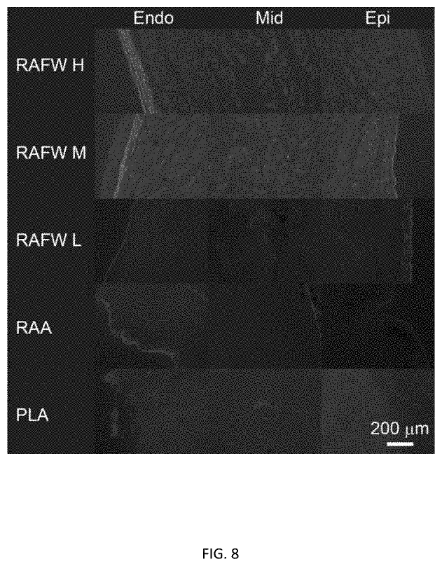

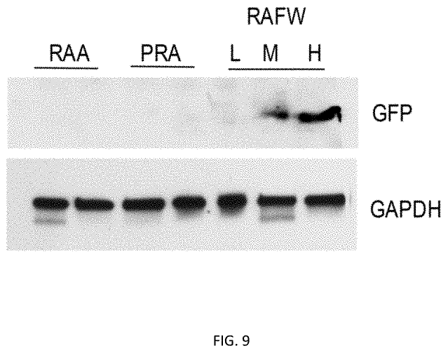

Injection ofGFP-expressingplasmid: In a third animal, 1.5 mg of Green Fluorescent Protein (GFP) expressing plasmid (under control of a CMV promoter) was diluted up to 20 ml and injected in the coronary sinus. Simultaneous electroporation was performed as described in the high right atrium (right atrial free wall) with a FirMap catheter, as described in the above paragraph for Coomassie blue. The gene injection and electroporation sequence was repeated three times. After 3 days, the animal was sacrificed and the heart removed for further analysis. The electroporated high right atrium (high and mid right atrial free wall) and non-electroporated right atrium (low right atrial free wall, right atrial appendage, posterior right atrium) and non-electroporated left atrium were examined for GFP expression using fluorescence microscopy and western blotting. As shown in FIG. 8 and FIG. 9, GFP expression was noted only in the electroporated parts of the right atrium i.e. high and mid right atrial free wall, with no evidence of GFP in non-electroporated right or left atrium. Furthermore, GFP expression was found to be transmural (i.e. epi to endocardial expression). These results demonstrate that it is possible to obtain robust gene expression in the atrium via this unique new ‘needleless’ method.

### REFERENCES

The following references are herein incorporated by reference in their entireties:
1. Korantzopoulos P, Kolettis T M, Galaris D and Goudevenos J A. The role of oxidative stress in the pathogenesis and perpetuation of atrial fibrillation. Int J Cardiol. 2007; 115:135-43.
2. Youn J Y, Zhang J, Zhang Y, Chen H, Liu D, Ping P, Weiss J N and Cai H. Oxidative stress in atrial fibrillation: an emerging role of NADPH oxidase. J Mol Cell Cardiol. 2013; 62:72-9.
3. Jeong E M, Liu M, Sturdy M, Gao G, Varghese S T, Sovari A A and Dudley S C, Jr. Metabolic stress, reactive oxygen species, and arrhythmia. J Mol Cell Cardiol. 2012; 52:454-63.

1. A method of delivering an agent to a target coronary tissue of a subject, comprising:
a) delivering the agent to a segment of the coronary vasculature of the subject; and
b) electroporating the target coronary tissue of the subject.

2. A method of claim 1, wherein the target coronary tissue is selected from the left atrium, the right atrium, the left ventricle, and the right ventricle of the subject.

3. The method of claim 1, wherein the segment of the coronary vasculature is selected from the aorta, the aortic root, the coronary artery, the coronary sinus, the vena cava, a pulmonary vein, a pulmonary artery, the brachiocephalic artery, the carotid artery, the subclavian artery, or the pericardial space.

4. The method of claim 3, wherein the segment of the coronary vasculature is selected from the aortic root, the coronary artery, and the coronary sinus.

5. The method of claim 1, wherein electroporation is performed prior to delivery of the agent, concurrently with delivery of the agent, and/or following delivery of the agent to the segment of the coronary vasculature ofthe subject.

6. The method of claim 1, wherein electroporating the target coronary tissue of the subject is performed by epicardial electroporation or endocardial electroporation.

7. The method of claim 1, wherein the agent comprises a therapeutic agent for the treatment of a cardiac disorder in the subject.

8. The method of claim 7, wherein the agent comprises anucleotide, an oligonucleotide, a protein, a peptide, a small molecule, or amacromolecule.

9. The method of claim 8, wherein the agent comprisesDNA.

10. The method of claim 7, wherein the cardiac disorder is a heart arrythmia.

11. The method of claim 10, wherein the cardiac disorder is atrial fibrillation or ventricular tachycardia.

12. A method of treating a cardiac disorder in a subject, comprising:
a) delivering an agent to a segment of the coronary vasculature of the subject; and
b) electroporating a target coronary tissue of the subject,

13. The method of claim 12, wherein the target coronary tissue is selected from the left atrium, the right atrium, the left ventricle, and the right ventricle of the subject.

14. The method of claim 13, wherein the cardiac disorder is a heart arrhythmia.

15. The method of claim 14, wherein the cardiac disorder is atrial fibrillation or ventricular tachycardia.

16. The method of claim 12, wherein the segment of the coronary vasculature is selected from the aorta, the aortic root, the coronary artery, the coronary sinus, the vena cava, a pulmonary vein, a pulmonary artery, the brachiocephalic artery, the carotid artery, the subclavian artery, or the pericardial space.

17. The method of claim 16, wherein the segment of the coronary vasculature is selected from the aortic root, the coronary artery, and the coronary sinus.

18. The method of claim 12, wherein electroporation is performed prior to delivery of the agent, concurrently with delivery of the agent, and/or following delivery of the agent to the segment of the coronary vasculature of thesubject.

19. The method of claim 12, wherein electroporating the target coronary tissue of the subject is performed by epicardial electroporation or endocardial electroporation.

20. The method of claim 12, wherein the agent comprises a nucleotide, an oligonucleotide, a protein, a peptide, a small molecule, or a macromolecule.

21. The method of claim 20, wherein the agent comprises DNA.

22. The method of claim 12, wherein the subject is a human.
